(19) United States
(12) Patent Application Publication
(10) Pub. No.: US 2021/0042915 A1
(43) Pub. Date: Feb. 11, 2021

(54) AUTOMATED MONITORING OF MEDICAL IMAGING PROCEDURES

(71) Applicant: MOBILEODT LTD., Tel Aviv (IL)

(72) Inventors: Amir Shlomo BERNAT, Moshav Mazor (IL); David LEVITZ, Tel Aviv (IL); Frank John BOLTON, Petah Tikva (IL); Kelwin FERNANDES, Porto (PT)

(21) Appl. No.: 16/964,303

(22) PCT Filed: Jan. 23, 2019
(86) PCT No.: PCT/IL2019/050098

§ 371 Date: Jul. 23, 2020

Related U.S. Application Data
(60) Provisional application No. 62/620,579, filed on Jan. 23, 2018.
(60) Provisional application No. 62/689,991, filed on Jun. 26, 2018.

(51) Int. Cl. G06T 7/00; G06T 7/73; G06T 7/90; G06N 3/08

(52) U.S. Cl. CPC G06T 7/0012; G06T 7/73; G06T 7/90; G06N 3/08; G06T 2207/20084; G06T 2207/10024; G06T 2207/30024; G06T 2207/20081; G06T 2207/10016

(57) ABSTRACT

A method for automated image capture of a body tissue in situ, comprising: providing an imaging device configured to transmit an image stream of a body tissue; and using at least one hardware processor for: receiving the image stream, identifying a medical accessory appearing in the image stream, and capturing multiple images of the body tissue, wherein (i) at least one of the images is captured before said identifying, and (ii) at least one of the images is captured at one or more specified times upon said identifying.

### CROSS REFERENCE TO RELATED APPLICATIONS

This application claims the benefit of priority to U.S. Provisional Patent Application No. 62/620,579 filed Jan. 23, 2018, entitled “AUTOMATED COLPOSCOPY IMAGE CAPTURE” and U.S. Provisional Patent Application No. 62/689,991 filed Jun. 26, 2018, entitled “AUTOMATED MONITORING OF MEDICAL IMAGING PROCEDURES”, the contents of which are incorporated herein by reference in their entirety

### FIELD OF THE INVENTION

The invention relates to the field of medical imaging systems.

### BACKGROUND

In the context of certain medical procedures, a contrast agent or medium is a substance applied to a tissue to enhance the visibility of certain structures or fluids within the body, in preparation for medical imaging. Several types of contrast media are in use in medical imaging, depending on the imaging modalities and where they are used. In areas such as colposcopy, an imaging device, such as a colposcope, may be used to identify visible clues suggestive of abnormal tissue. The imaging device may obtain images of the observed area, while contrast media, such as diluted acetic acid, may be applied to the observed tissue, to improve visualization of abnormalities. The effect of the acetic acid, known as “aceto-whitening,” may peak at specified times after its application, depending, for example, on the type and grade of the abnormality, lesion, or growth in question. Thus, it is important to obtain images of the observed area at one or more specific points in time during the observation, for an accurate and effective diagnosis.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

There is provided, in some embodiments, a method for automated image capture of a body tissue in situ, comprising: providing an imaging device configured to transmit an image stream of a body tissue; and using at least one hardware processor for: receiving the image stream, identifying a medical accessory appearing in the image stream, and capturing multiple images of the body tissue, wherein: (i) at least one of the images is captured before said identifying, and (ii) at least one of the images is captured at one or more specified times upon said identifying.

There is also provided, in some embodiments, a system for automated image capture of a body tissue in situ, comprising an imaging device configured to transmit an image stream of a body tissue; at least one hardware processor; and a non-transitory computer-readable storage medium having stored thereon program instructions, the program instructions executable by the at least one hardware processor, to provide an imaging device configured to transmit an image stream of a body tissue; receive the image stream, identify a medical accessory appearing in the image stream, and capture multiple images of the body tissue, wherein: (i) at least one of the images is captured before said identifying, and (ii) at least one of the images is captured at one or more specified times upon said identifying.

There is further provided, in some embodiments, a computer program product, the computer program product comprising a non-transitory computer-readable storage medium having program code embodied therewith, the program code executable by at least one hardware processor to provide an imaging device configured to transmit an image stream of a body tissue; receive the image stream, identify a medical accessory appearing in the image stream, and capture multiple images of the body tissue, wherein: (i) at least one of the images is captured before said identifying, and (ii) at least one of the images is captured at one or more specified times upon said identifying.

In some embodiments, said body tissue is a cervical tissue.

In some embodiments, said medical accessory is a swab used for applying a contrast agent to the body tissue.

In some embodiments, said specified times are determined based, at least in part, on a type of said contrast agent.

In some embodiments, said contrast agent is acetic acid, and said specified times are within the range of 15-600 seconds.

In some embodiments, said range is 90-150 seconds.

In some embodiments, said identifying comprises one or more of: determining a presence of said medical accessory in said image stream for a specified period of time; determining a removal of said medical accessory from said image stream; determining a distance of said medical accessory from the imaging device; determining a specified position of said medical accessory in relation to said body tissue; determining a specified orientation of said medical accessory in relation to said body tissue; identifying a specified object appearing in the image stream simultaneously with said medical accessory; tracking said medical accessory to identify a specified action thereof; and detecting contact between said medical accessory and said body tissue.

In some embodiments, said identifying is executed by a convolutional neural network (CNN) classifier. In some embodiments, said CNN classifier is trained on a set of labelled images of one of more types of said medical accessory shown against a background of cervical tissue.

In some embodiments, said specified times are determined based on user selection.

In some embodiments, at least some of said multiple images are captured during a specified time period before said specified times, and wherein at least some of said multiple images are captured during a specified time period after said specified times.

In some embodiments, said capturing comprises capturing one or more of a continuous video sequence, a time-lapse video sequence, and a sequence of images.

In some embodiments, said capturing further comprises selecting an image from the sequence of images based at least in part on an image quality parameter.

In some embodiments, said capturing further comprises performing one or more adjustments to said image stream, and wherein said adjustments are selected from the group consisting of: magnification, focus, color filtering, polarization, glare removal, white balance, exposure time, gain (ISO), and gamma.

In some embodiments, the method further comprises providing, and the program instructions are further executable to provide, a user interface module comprising one or more of: a display, a speaker, a control panel, a microphone, and a printer. In some embodiments, said image stream is presented on said display in real time.

In some embodiments, said capturing further comprises one of more of: storing said at least one of said one or more images on a storage medium; annotating at least one of said one or more images with specified information; transmitting at least one of said one or more images via a network connection; and printing a hard copy of at least one image of said one or more images.

In some embodiments, the method further comprises providing, and the program instructions are further executable to provide, a light source configured to provide illumination of said body tissue.

In some embodiments, said imaging device is configured to be mounted on a swingarm.

There is also provided, in an embodiment, a method comprising: receiving an image stream of a cervix during an examination period, wherein said cervix undergoes, during said examination period, at least one of: (i) removal of a mucus layer covering at least part of said cervix, and (ii) application of a contrast agent to said cervix; and detecting in said image stream at least one of: (iii) an incomplete removal of said mucus layer, (iv) a reversal of an effect provoked in said cervix by said contrast agent, and (v) a concentration level of said contrast agent, wherein said detecting is based, at least in part, on measuring, in said image stream, temporal changes in at least one optical property of said tissue sample.

There is further provided, in an embodiment, a system for automated image capture of a body tissue in situ, comprising at least one hardware processor; and a non-transitory computer-readable storage medium having stored thereon program instructions, the program instructions executable by the at least one hardware processor, to: receive an image stream of a cervix during an examination period, wherein said cervix undergoes, during said examination period, at least one of: (i) removal of a mucus layer covering at least part of said cervix, and (ii) application of a contrast agent to said cervix; and detect in said image stream at least one of: (iii) an incomplete removal of said mucus layer, (iv) a reversal of an effect provoked in said cervix by said contrast agent, and (v) a concentration level of said contrast agent, wherein said detecting is based, at least in part, on measuring, in said image stream, temporal changes in at least one optical property of said tissue sample.

There is further provided, in an embodiment, a computer program product, the computer program product comprising a non-transitory computer-readable storage medium having program code embodied therewith, the program code executable by at least one hardware processor to: receive an image stream of a cervix during an examination period, wherein said cervix undergoes, during said examination period, at least one of: (i) removal of a mucus layer covering at least part of said cervix, and (ii) application of a contrast agent to said cervix; and detect in said image stream at least one of: (iii) an incomplete removal of said mucus layer, (iv) a reversal of an effect provoked in said cervix by said contrast agent, and (v) a concentration level of said contrast agent, wherein said detecting is based, at least in part, on measuring, in said image stream, temporal changes in at least one optical property of said tissue sample.

In some embodiments, said detecting comprises first identifying in said image stream boundaries of said cervix.

In some embodiments, said identifying is based, at least in part, on tissue sample color.

In some embodiments, said identifying is based, at least in part, on executing one or more machine learning algorithms selected from the group consisting of convolutional neural network (CNN) classifiers and support vector machine (SVM) classifiers.

In some embodiments, said contrast agent is acetic acid and said effect is acetowhitening.

In some embodiments, said contrast agent is Lugol's iodine and said effect is associated with an uptake of iodine in glycogen-containing tissue areas.

In some embodiments, said detecting of said incomplete removal of said mucus layer comprises comparing at least a first said image captured before said removal with at least a second said image captured after said removal, and wherein said comparison is based, at least in part, on pixel values in said first and second images meeting a dissimilarity threshold.

In some embodiments, said detecting further comprises issuing an alert associated with said incomplete removal, to a clinician executing said examination.

In some embodiments, said alert comprises an indication as to the location of one or more areas within said image stream identified as associated with said incomplete removal of said mucus layer, wherein said indication comprises at least one of: displaying an outline around said one or more areas, displaying a box surrounding said one or more areas, displaying an arrow pointing to the said one or more areas, displaying a zoomed-in view of said one or more areas, and/or displaying an indication of one or more values associated with said one or more areas.

In some embodiments, said detecting of said reversal of said effect further comprises determining a time of said application of said contrast agent, based, at least in part, on identifying said one or more swabs appearing in the image stream.

In some embodiments, said measuring takes place during a specified period after said determining.

In some embodiments, said measuring further comprises calculating, during said period, at least one parameter of said temporal changes in said effect, wherein said at least one parameter is selected from the group consisting of: a maximum value, a time period required for reaching said maximum value, an integral of a curve describing temporal changes in said value, a rate of increase in said value to said maximum value, and a rate of decrease in said value from said maximum value.

In some embodiments, the method further comprises associating, and the program instructions are further executable to associate, at least one said parameter with a tissue condition selected from the group consisting of: normal tissue, inflamed tissue, cervical neoplasia, HPV infection, dysplasia, pathological tissue, precancerous tissue, and cancerous tissue.

In some embodiments, the method further comprises determining, and the program instructions are further executable to determine, at least one of (i) a need for one or more additional said applications of said contrast agent, and (ii) a need to adjust said concentration level of said contrast agent, based, at least on part, on said associating.

In some embodiments, said determining further comprises issuing one or more alerts associated with said determining, to a clinician executing said examination.

In some embodiments, said capturing comprises using at least one imaging device configured to detect at least one of RGB (red-green-blue), monochrome, ultraviolet (UV), near infrared (NIR), and short-wave infrared (SWIR) spectral data.

In some embodiments, said at least one imaging device comprises a digital imaging sensor selected from the group consisting of: complementary metal-oxide-semiconductor (CMOS), charge-coupled device (CCD), Indium gallium arsenide (InGaAs), and polarization-sensitive sensor element.

In some embodiments, said capturing comprises capturing one or more of a continuous video sequence, a time-lapse video sequence, and a sequence of images.

In some embodiments, said capturing further comprises performing one or more adjustments to said images, and wherein said adjustments are selected from the group consisting of: magnification, focus, color filtering, polarization, and glare removal.

There is also provided, in an embodiment, a method comprising: receiving an image stream depicting at least a cervix; detecting, in the image stream, at least one of: (i) one or more swabs, (ii) an incomplete removal of a mucus layer from the cervix, (iii) a reversal of an effect provoked in the cervix by a contrast agent, and (iv)a concentration level of the contrast agent; issuing a notification immediately upon the detection of at least one of (ii)-(iv); capturing images of the cervix (a) before an application of the contrast agent by the one or more swabs, and (b) at specified times after a successful application of the contrast agent by the swab, wherein the successful application is determined based on detection or lack of detection of at least one of (ii)-(iv).

There is further provided, in an embodiment, a system for automated image capture of a body tissue in situ, comprising at least one hardware processor; and a non-transitory computer-readable storage medium having stored thereon program instructions, the program instructions executable by the at least one hardware processor, to: receive an image stream depicting at least a cervix; detect, in the image stream, at least one of: (i) one or more swabs, (ii) an incomplete removal of a mucus layer from the cervix, (iii) a reversal of an effect provoked in the cervix by a contrast agent, and (iv)a concentration level of the contrast agent; issue a notification immediately upon the detection of at least one of (ii)-(iv); capture images of the cervix (a) before an application of the contrast agent by the one or more swabs, and (b) at specified times after a successful application of the contrast agent by the swab, wherein the successful application is determined based on detection or lack of detection of at least one of (ii)-(iv).

There is further provided, in an embodiment, a computer program product, the computer program product comprising a non-transitory computer-readable storage medium having program code embodied therewith, the program code executable by at least one hardware processor to: receive an image stream depicting at least a cervix; detect, in the image stream, at least one of: (i) one or more swabs, (ii) an incomplete removal of a mucus layer from the cervix, (iii) a reversal of an effect provoked in the cervix by a contrast agent, and (iv)a concentration level of the contrast agent; issue a notification immediately upon the detection of at least one of (ii)-(iv); capture images of the cervix (a) before an application of the contrast agent by the one or more swabs, and (b) at specified times after a successful application of the contrast agent by the swab, wherein the successful application is determined based on detection or lack of detection of at least one of (ii)-(iv).

In some embodiments, said specified times are determined based, at least in part, on a type of said contrast agent.

In some embodiments, said contrast agent is acetic acid, and said specified times are within the range of 15-600 seconds.

In some embodiments, said range is 90-150 seconds.

In some embodiments, said detecting of one or more swabs comprises one or more of: determining a presence of said swab in said image stream for a specified period of time; determining a removal of said swab from said image stream; determining a distance of said swab from the imaging device; determining a specified position of said swab in relation to said cervix; determining a specified orientation of said swab in relation to said cervix; identifying a specified object appearing in the image stream simultaneously with said swab; tracking said swab to identify a specified action thereof; and detecting contact between said swab and said cervix.

In some embodiments, said detecting is executed by a convolutional neural network (CNN) classifier.

In some embodiments, said CNN classifier is trained on a set of labelled images of one of more types of said swab shown against a background of cervical tissue.

In some embodiments, said specified times are determined based on user selection.

In some embodiments, at least some of said images are captured during a specified time period before said specified times, and wherein at least some of said multiple images are captured during a specified time period after said specified times.

In some embodiments, said capturing comprises capturing one or more of a continuous video sequence, a time-lapse video sequence, and a sequence of images.

In some embodiments, said capturing further comprises selecting an image from the sequence of images based at least in part on an image quality parameter.

In some embodiments, said capturing further comprises performing one or more adjustments to said image stream, and wherein said adjustments are selected from the group consisting of: magnification, focus, color filtering, polarization, glare removal, white balance, exposure time, gain (ISO), and gamma.

In some embodiments, the method further comprises providing, and the program instructions are further executable to provide, a user interface module comprising one or more of: a display, a speaker, a control panel, a microphone, and a printer.

In some embodiments, said image stream is presented on said display in real time.

In some embodiments, said capturing further comprises one of more of: storing said at least one of said one or more images on a storage medium; annotating at least one of said one or more images with specified information; transmitting at least one of said one or more images via a network connection; and printing a hard copy of at least one image of said one or more images.

In some embodiments, the method further comprises providing, and the program instructions are further executable to provide, a light source configured to provide illumination of said cervix.

In some embodiments, said detecting comprises first identifying in said image stream boundaries of said cervix.

In some embodiments, said identifying is based, at least in part, on tissue sample color.

In some embodiments, said identifying is based, at least in part, on executing one or more machine learning algorithms selected from the group consisting of convolutional neural network (CNN) classifiers and support vector machine (SVM) classifiers.

In some embodiments, said contrast agent is acetic acid and said effect is acetowhitening.

In some embodiments, said contrast agent is Lugol's iodine and said effect is associated with an uptake of iodine in glycogen-containing tissue areas.

In some embodiments, said detecting of said incomplete removal of said mucus layer comprises comparing at least a first said image captured before said removal with at least a second said image captured after said removal, and wherein said comparison is based, at least in part, on pixel values in said first and second images meeting a dissimilarity threshold.

In some embodiments, said detecting further comprises issuing an alert associated with said incomplete removal, to a clinician executing said examination.

In some embodiments, said alert comprises an indication as to the location of one or more areas within said image stream identified as associated with said incomplete removal of said mucus layer, wherein said indication comprises at least one of: displaying an outline around said one or more areas, displaying a box surrounding said one or more areas, displaying an arrow pointing to the said one or more areas, displaying a zoomed-in view of said one or more areas, and/or displaying an indication of one or more values associated with said one or more areas.

In some embodiments, said detecting of said reversal of said effect further comprises determining a time of said application of said contrast agent, based, at least in part, on identifying a medical swab appearing in the image stream.

In some embodiments, the method further comprises determining, and the program instructions are further executable to determine, at least one of (i) a need for one or more additional said applications of said contrast agent, and (ii) a need to adjust said concentration level of said contrast agent, based, at least on part, on said associating.

In some embodiments, said determining further comprises issuing one or more alerts associated with said determining, to a clinician executing said examination.

In some embodiments, said capturing comprises using at least one imaging device configured to detect at least one of RGB (red-green-blue), monochrome, ultraviolet (UV), near infrared (NIR), and short-wave infrared (SWIR) spectral data.

In some embodiments, said at least one imaging device comprises a digital imaging sensor selected from the group consisting of: complementary metal-oxide-semiconductor (CMOS), charge-coupled device (CCD), Indium gallium arsenide (InGaAs), and polarization-sensitive sensor element.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1 illustrates an automated system for time-dependent image capture during medical procedures, according to an embodiment;

FIG. 2 is a flowchart of the functional steps in a method for automated time-dependent image capture during medical procedures, according to an embodiment;

FIG. 3A shows a reference image of a cervix captured through an opening of a speculum at a pre-cleaning stage;

FIG. 3B shows a leukoplakia area in a pre-acetowhitening and a post-acetowhitening stage;

FIG. 3C illustrates comparing pre- and post-cleaning images to each another, according to an embodiment;

FIG. 3D illustrates detecting areas which have not been thoroughly cleaned of mucus in dpatients with an IUD, according to an embodiment;

FIGS. 4A-4B schematically illustrate an exemplary application of a system for time-dependent image capture during medical procedures, according to an embodiment;

FIG. 5 demonstrates an exemplary analysis of pre- and post-acetowhitening images in the HSV color space;

FIG. 6 shows images converted into the HSV and the La*b* color spaces; and

FIG. 7 shows a cervical area which has undergone an acetowhitening response corresponding to a tissue pathology.

### DETAILED DESCRIPTION

A system and a method are disclosed herein to enable automated time-dependent image capture during medical procedures, based on computer vision object recognition. The present system and method comprise an imaging system configured to automatically capture one or more images of a region of the body at specific time intervals, based upon recognition of a specified triggering event, which may be a medical accessory or instrument introduced into the field of vision of an imaging system.

In some embodiments, the present method further provides for the automated monitoring and detection of improper and/or incomplete execution of a medical examination procedure involving the application of a contrast agent.

In some embodiments, the present invention may be configured for alerting a clinician regarding improper and/or incomplete surface area preparation, inconsistent contrast agent application, and/or inadequate contrast agent concentration levels, as well as steps that can be taken to correct these improper/incomplete steps.

Accordingly, the present invention may help to promote more consistent, accurate and reliable imaging results. The increased accuracy and reliability of the results may offer a reduced risk of missing high-grade disease, added reassurance in treatment decisions, and elimination of unnecessary treatments. In addition, by providing for greater consistency in imaging results, the present invention may facilitate greater use of computerized image evaluation applications, thereby reducing the reliance on medical experts and increasing overall efficiency.

As used in here, the terms “image” or “image frame” refer to any image or portion of an image that includes a two-, three, or higher-dimensional representation of a physical object or a scene.

The term “imaging device” is broadly defined as any device that captures images and represents them as data. Imaging devices may be optic-based, such as image sensors, but may also include depth sensors, radio frequency imaging, ultrasound imaging, infrared imaging, and the like.

The terms “object recognition” and “object classification” refer to recognition and classification of objects in a digital image using computer vision.

Exemplary embodiments of the present system and method may be employed in connection with medical procedures involving the use of a contrast agent or medium. Contrast agents are applied to an area of the body under examination, to enhance the visibility of certain structures or fluids. The enhancement effect of certain contrast agents may peak at specific times after the application thereof. Accordingly, for optimal diagnosis, it is advantageous to capture one or more images of the affected bodily tissue at the predetermined specific peak times after the application of the contrast agent.

In certain medical procedures, an appropriate contrast agent is applied to an area of the body under observation, to enhance the visibility of certain tissue structures. The contrast agent interacts selectively with pathological tissue to alter its optical characteristics, thus enhancing the contrast between lesion and healthy tissue. These optical alterations can in turn be detected in vivo, by exploiting the light-tissue interaction phenomena. The measurement and analysis of the characteristics of the remitted light from the tissue can also provide information about the presence of different molecules, or about the various structural and functional changes occurring during the progress of the disease, thus providing a means for the in-vivo identification and grading of the lesion.

The following discussions will focus on the medical procedures of colposcopy, where a colposcope is used to identify visible clues suggestive of abnormal tissue in the cervix of the uterus. However, in addition to colposcopy, other types of diagnostic and therapeutic treatments may benefit from improved consistency and reliability of visualization in results, including, but not limited to, general surgery (endoscopy and laparoscopy), gastroenterology, ear nose and throat (ENT), urology, forensic medicine (i.e., evidence collection from patients who are criminal victims or suspects), and visualization of injuries in general (including diabetic ulcers).

Current imaging systems are, for the most part, manually operated or require timely input from an operator. For example, in colposcopy, a cervical imaging device, such as a colposcope, may be positioned on a tripod, a mount, or a swingarm. The imaging device may be trained on the observed area in the cervix, e.g., through a vaginal speculum, so as to acquire one or more images thereof. A medical professional administering the procedure may need to hold and maneuver the colposcope with one hand, apply the contrast medium to an area of the cervix with a cotton swab using the other hand, then start and observe a countdown timer, and manually operate the imaging device upon the expiration of one or more desired time intervals. Needless to say, such manual operation is cumbersome and ties up the hands and attention of the medical professional, and as such it is prone to distractions and errors, which may affect the accuracy of the results and ultimate diagnosis. For example, because one hand of the physician administering the procedure is occupied handling the colposcope, and the other conducting the swabbing, the start of the timer function will, by necessity, be slightly delayed, because it depends on the physician completing the swabbing, then retrieving the cotton swab back through the speculum, discarding it, and only then operating the timer. Alternatively, such procedure may require the presence of additional personnel, such as a nurse or assistant, to operate the timer and imaging device upon instructions from the physician, thus tying up valuable resources.

Accordingly, in an exemplary embodiment of the present invention, a system and method are disclosed for use in cervico-vaginal imaging, wherein an imaging device is configured to automatically capture and/or store one or more images at specified time points upon the occurrence of a specified triggering event, such as the detection of a medical applicator (e.g., a swab), in the field of view of the imaging device. The increased accuracy of the results may offer a reduced risk of missing high grade disease, added reassurance in treatment decisions, and reduced risk of unnecessary treatment.

Colposcopic examination is routinely used as a diagnostic tool for in vivo, non-invasive identification of abnormal areas of the cervix. A colposcope functions as a lighted optical magnification device to visualize and/or capture images which provide a general impression of the surface architecture of the cervical tissue, as well as certain vascular patterns that may indicate the presence of more advanced precancerous or cancerous lesions. During colposcopy, a contrast agent, such as diluted acetic acid (at 3-5%), Lugol's iodine, and/or Toluidine blue, is applied to the entire cervix to highlight areas with a high risk of dysplasia/precancer. Such areas may react to the application of the contrast agent by undergoing a process of aceto-whitening, which correlates with the higher nuclear density of precancerous lesions.

During examination, the cervical tissue in the images is evaluated in a time-resolved manner according to several criteria, such as the morphology of the lesion's margins, the vascular pattern of abnormal epithelium, and the degree of staining after application of the contrast agent. Colposcopic grading is based on visual examination, and the detected lesions are classified according to empirically qualitative scales. For example, high-grade (level 2-3) cervical intraepithelial neoplasia (CIN) are associated with thick, dense, dull, opaque or greyish-white aceto-whitened areas with well-demarcated, regular margins, which sometimes may be raised and rolled out. However, considerable skill and prolonged evaluation may be required to differentiate between low-grade CIN, immature squamous metaplasia, and inflammatory lesions.

In all cases, the effectiveness of colposcopy greatly depends on properly following colposcopy procedure. Some of the issues which may arise in this regard include improper and/or incomplete surface preparation, improper or insufficient administration of the contrast agent, and/or inadequate concentration of the contrast agent solution. In many cases, this may be the result of lack of training or insufficient clinical experience on part of the clinician performing the examination. In this regard, industry experts have estimated that clinicians may require performing between 25-100 cases with a preceptor, before their training is complete (see, e.g., “Colposcopy can enhance your diagnostic skills”, Relias AHC Media, Jan. 1, 1998). However, in developing areas of the world, where examinations are sometimes conducted in field conditions, experienced or properly-trained staff may be difficult to recruit.

Improper surface area preparation may involve, e.g., incomplete removal of the mucus layer of the cervix. Typically, colposcopy preparation begins with the removal of excess mucus and/or other contaminants, such as blood, from the cervix with dry or saline-soaked cotton swabs. This allows initial visualization of any obvious findings indicative of invasive cancer such as erosion, surface contour abnormality, leukoplakia, or exophytic lesions. Some abnormal vascular patterns are more visible before the application of acetic acid, with the use of a red-free (i.e., green) filter. In addition, it should be noted that medical imaging of in-vivo diagnostic inspections requires the illumination and imaging rays to travel along the same optical path, through the cavities of the body. In some cases, the tissue's surface reflection figures substantially in the resulting image as optical noise in the diagnostic signal, thus degrading substantially the perceived contrast between agent-responsive and agent non-responsive tissue areas. This problem is especially significant in epithelial tissues, such as the cervix, larynx, oral cavity, etc., which are covered by fluids such as mucus and saliva. Surface reflection also obstructs the detection and measurement of the alterations in the tissue's optical properties, provoked after the administration of contrast agents which enhance the optical contrast between normal and pathologic tissue. More specifically, when a contrast agent selectively alters the scattering characteristics of the pathological tissue, the strong surface reflection that takes place in both pathological (agent-responsive) and normal (agent non-responsive) tissue areas saturates the camera, and hides the diagnostic signal that originates from the interaction of the agent with the subsurface features of the tissue.

Another potential issue relates to the fact that the visual effect of aceto-whitening is a dynamic phenomenon which should preferably be assessed in a time-resolved manner and not statically. Generally, the aceto-whitening effect increases progressively, until it peaks at about 120 seconds, after which time it begins to reverse and decay (however, different colposcopy guidelines instruct differently with respect to peak effect timing). However, in several cases of early pathological conditions, the phenomenon of temporary staining after administering the agent is short-lasting and thus the clinician is not able to detect the provoked alterations and even more so, to assess their intensity and extent. Thus, for example, more contrast agent may need to be applied at specified intervals, to maintain the acetowhitening effect for a longer period of time. These variations often result in the downgrading of the diagnostic value and usefulness of the procedure.

The quality, consistency and reliability of colposcopy imaging may be especially significant when images are sent for assessment by an expert at a remote location (e.g., when images are taken in the field in developing countries), and/or when relying on automated or semi-automated image analysis applications to analyze colposcopy images. Inconsistent or unreliable images, which are the result of an improperly conducted examination, may raise the issues of false positives and false negatives in the diagnosis. This may require routine double-checking by a medical expert, which runs counter to the purpose of using automated applications in the first place. In addition, poor diagnostic results may require the patient to return in order to be subjected again to a new colposcopy procedure, which again causes a waste of time and valuable resources.

Accordingly, a potential advantage of the present invention is in that it provides for a real-time, automated monitoring and detection of improper and/or incomplete colposcopy procedure, including alerting the clinician in cases of incomplete and/or improper area cleaning, the need for one or more reapplications of contrast agent, and/or the need for monitoring of contrast agent concentration levels.

### Exemplary System of the Present Invention

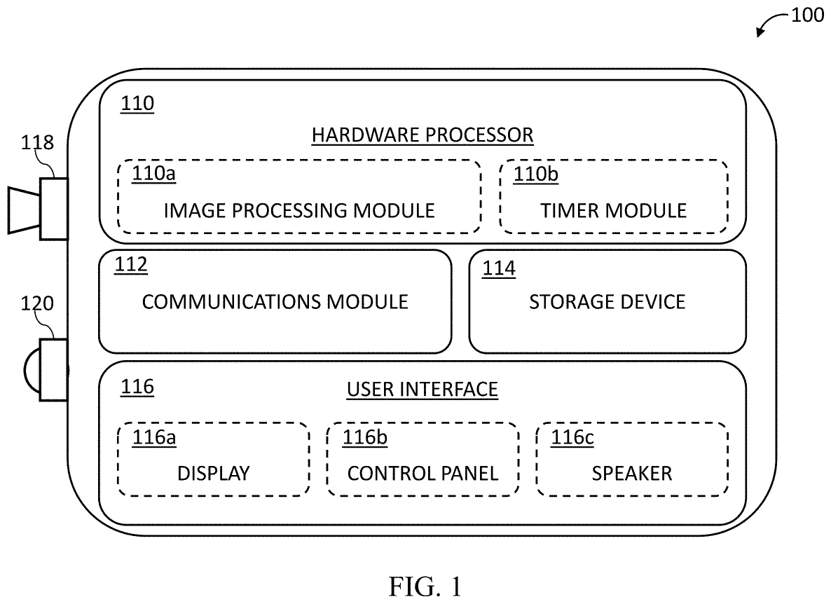

FIG. 1 is a block diagram of an exemplary system 100 for automated monitoring of medical imaging procedures involving the application of a contrast agent. System 100 as described herein is only an exemplary embodiment of the present invention, and in practice may have more or fewer components than shown, may combine two or more of the components, or a may have a different configuration or arrangement of the components. The various components of system 100 may be implemented in hardware, software or a combination of both hardware and software. In various embodiments, system 100 may comprise a dedicated hardware device, such as a mobile device, a cellphone, a digital camera, and the like, or may form an addition to or extension of an existing medical device, such as a colposcope, a cervicography imaging device, a cervical imaging probe (which may have various shapes and sizes, e.g., a tampon form factor probe), and the like.

In some embodiments, system 100 may comprise a hardware processor 110, communications module 112, memory storage device 114, user interface 116, imaging device 118, and a light source 120. System 100 may store in a non-volatile memory thereof, such as storage device 114, software instructions or components configured to operate a processing unit (also “hardware processor,” “CPU,” or simply “processor), such as hardware processor 110. In some embodiments, the software components may include an operating system, including various software components and/or drivers for controlling and managing general system tasks (e.g., memory management, storage device control, power management, etc.) and facilitating communication between various hardware and software components.

In some embodiments, non-transient computer-readable storage device 114 (which may include one or more computer readable storage mediums) is used for storing, retrieving, comparing, and/or annotating captured frames. Image frames may be stored on storage device 114 based on one or more attributes, or tags, such as a time stamp, a user-entered label, or the result of an applied image processing method indicating the association of the frames, to name a few.

The software instructions and/or components operating hardware processor 110 may include instructions for receiving and analyzing multiple frames captured by imaging device 118. For example, hardware processor 110 may comprise image processing module 110a, which receives one or more images and/or image streams from imaging device 118 and applies one or more image processing algorithms thereto. In some embodiments, image processing module 110a comprises one or more algorithms configured to perform object recognition and classification in images captured by imaging device 118, using any suitable image processing or feature extraction technique. For some embodiments, image processing module 110a can simultaneously receive and switch between multiple input image streams to multiple output devices while providing image stream processing functions on the image streams. The incoming image streams may come from various medical or other imaging devices. The image streams received by the image processing module 110a may vary in resolution, frame rate (e.g., between 15 and 35 frames per second), format, and protocol according to the characteristics and purpose of their respective source device. Depending on the embodiment, the image processing module 110a can route image streams through various processing functions, or to an output circuit that sends the processed image stream for presentation, e.g., on a display 116a, to a recording system, across a network, or to another logical destination. In image processing module 110a, the image stream processing algorithm may improve the visibility and reduce or eliminate distortion, glare, or other undesirable effects in the image stream provided by an imaging device. An image stream processing algorithm may reduce or remove fog, smoke, contaminants, or other obscurities present in the image stream. The types of image stream processing algorithms employed by the image stream processing module 110a may include, for example, a histogram equalization algorithm to improve image contrast, an algorithm including a convolution kernel that improves image clarity, and a color isolation algorithm. The image stream processing module 110a may apply image stream processing algorithms alone or in combination.

Image processing module 110a may also facilitate logging or recording operations with respect to an image stream. According to some embodiments, the image processing module 110a enables recording of the image stream with a voice-over or a bookmark, or capturing of frames from an image stream (e.g., drag-and-drop a frame from the image stream to a window). Some or all of the functionality of the image processing module 110a may be facilitated through an image stream recording system or an image stream processing system.

Hardware processor 110 may also comprise timing module 110b, which may provide timing capabilities using one or more timers, clocks, stop-watches, alarms, and/or the like, that trigger various functions of system 100, such as image capture. Such timers, stop-watches and clocks may also be added and displayed over the image stream through user interface 116. For example, the timing module 110b may allow a user to add to the display a countdown timer, e.g., in association with a surgical or diagnostic and/or other procedure. A user may be able to select from a list of pre-defined countdown timers, which may have been pre-defined by the user. In some variations, a countdown timer may be displayed on a display 116a, bordering or overlaying the image stream.

In some embodiments, system 100 comprises a communications module (or a set of instructions), a contact/motion module (or a set of instructions), a graphics module (or a set of instructions), a text input module (or a set of instructions), a Global Positioning System (GPS) module (or a set of instructions), voice recognition and/or and voice replication module (or a set of instructions), and one or more applications (or sets of instructions).

For example, a communications module 112 may connect system 100 to a network, such as the Internet, a local area network, a wide area network and/or a wireless network. Communications module 112 facilitates communications with other devices over one or more external ports, and also includes various software components for handling data received by system 100. For example, communications module 112 may provide access to a patient medical records database, e.g., from a hospital network. The content of the patient medical records may comprise a variety of formats, including images, audio, video, and text (e.g., documents). In some embodiments, system 100 may access information from a patient medical record database and provide such information through the user interface 116, presented over the image stream on display 116a. communications module 112 may also connect to a printing system configured to generate hard copies of images captured from an image stream received, processed, or presented through system 100.

In some embodiments, a user interface 116 of system 100 comprises a display monitor 116a for displaying images, a control panel 116b for controlling system 100, and a speaker 116c for providing audio feedback. In some variations, display 116a may be used as a viewfinder and/or a live display for either still and/or video image acquisition by imaging device 118. The image stream presented by display 116a may be one originating from imaging device 118. Display 116a may be a touch-sensitive display. The touch-sensitive display is sometimes called a “touch screen” for convenience, and may also be known as or called a touch-sensitive display system. Touch-sensitive display may be configured to detect commands relating to activating or deactivating particular functions of system 100. Such functions may include, without limitation, image stream enhancement, management of windows for window-based functions, timers (e.g., clocks, countdown timers, and time-based alarms), tagging and tag tracking, image stream logging, performing measurements, two-dimensional to three- dimensional content conversion, and similarity searches.

Imaging device 118 is broadly defined as any device that captures images and represents them as data. Imaging devices may be optic-based, but may also include depth sensors, radio frequency imaging, ultrasound imaging, infrared imaging, and the like. In some embodiments, imaging device 118 may be configured to detect RGB (red-green-blue) spectral data. In other embodiments, imaging device 118 may be configured to detect at least one of monochrome, ultraviolet (UV), near infrared (NIR), and short-wave infrared (SWIR) spectral data. In some embodiments, imaging device 118 comprises a digital imaging sensor selected from the group consisting of silicon-based detectors, complementary metal-oxide-semiconductor (CMOS), charge-coupled device (CCD), Indium gallium arsenide (InGaAs), and polarization-sensitive sensor element. In some embodiments, imaging device 118 is configured to capture images of a tissue sample in vivo, along a direct optical path from the tissue sample. In other embodiments, imaging device 118 is coupled to a light guide, e.g., a fiber optic light guide, for directing a reflectance and/or fluorescence from the tissue sample to imaging device 118. Imaging device 118 may further comprise, e.g., zoom, magnification, and/or focus capabilities. Imaging device 118 may also comprise such functionalities as color filtering, polarization, and/or glare removal, for optimum visualization. Imaging device 118 may further include an image stream recording system configured to receive and store a recording of an image stream received, processed, and/or presented through system 100. In some embodiments, imaging device 118 may be configured to capture a plurality of RGB images, wherein imaging device 118 and/or image processing module 110a may be configured for changing the ratio of individual RGB channels, for example, by amplifying at least one of the green channel and the red channel.

According to an embodiment, a light source 120 is configured to emit light at one or more spectral bands. In some embodiment, light source 120 produces purple light having a wavelength selected from the range 390-430 nanometer (nm). Due to its short wavelength, purple light has the highest spatial resolution of any spectral band within the visible spectrum. However, because it is still part of the visible spectrum, it can be captured by many common imaging devices, such as regular digital RGB (red-green-blue) cameras and monochrome cameras. Purple light also has the highest reduced scattering coefficientₛ′) of all the visible wavelengths, resulting in a very shallow penetration depth into the tissue, and relatively high amounts of light that is diffusely reflected from the tissue. All of these attributes make purple light particularly beneficial in diagnostic procedures involving visualization through contrast agents that are scattering-based, such as acetic acid. In addition, the short wavelength of purple light makes it useful for exciting fluorescence in some molecules in tissue.

In other embodiments, system 100 may comprise additional illumination sources, wherein a tissue sample may be illuminated with purple light in combination with one or more additional lights transmitted simultaneously or sequentially with the purple light, based, e.g., on user selection. For example, system 100 may include one or more light sources having wavelengths selected from the ranges 450-500 nm (the blue wavelength range); 500-570 nm (the green wavelength region); 585-720 nm (the red wavelength region); 900-3000 nm (the near-infrared and shortwave infrared wavelength regions); and/or 100-390 nm (the ultraviolet wavelength region). In yet other embodiments, system 100 may include polarization difference imaging (PDI) and/or structured illumination techniques, such as Spatial Frequency Domain Imaging (SFDI).

Light source 120 may comprise, e.g., any suitable light source selected from the group consisting of incandescent light bulb, light emitting diode (LED), laser diode, light emitter, bi-spectral emitter, dual spectral emitter, photodiode, and semiconductor die. In some embodiments, light source 120 is configured to illuminate the tissue sample directly. In other embodiments, light source 120 is configured to transmit illumination through a suitable conduit, e.g., an optical fiber cable, for higher focus and intensity of illumination.

System 100 may further comprise, e.g., light collection optics; beam splitters and dichroic mirrors to split and direct a desired portion of the spectral information towards more than one imaging device; and/or multiple optical filters having different spectral transmittance properties, for selectively passing or rejecting passage of radiation in a wavelength-, polarization-, and/or frequency-dependent manner.

In some embodiments, system 100 includes one or more user input control devices, such as a physical or virtual joystick, mouse, and/or click wheel. In other variations, system 100 comprises one or more of a peripherals interface, RF circuitry, audio circuitry, a microphone, an input/output (I/O) subsystem, other input or control devices, optical or other sensors, and an external port. System 100 may also comprise one or more sensors, such a proximity sensors and/or accelerometers. Each of the above identified modules and applications correspond to a set of instructions for performing one or more functions described above. These modules (i.e., sets of instructions) need not be implemented as separate software programs, procedures or modules, and thus various subsets of these modules may be combined or otherwise re-arranged in various embodiments.

In some embodiments, system 100 is mounted on a stand, a tripod and/or a mount, which may be configured for easy movability and maneuvering (e.g., through caster wheels). In some embodiments, the stand may incorporate a swingarm or another type of an articulated arm. In such embodiments, imaging device 118 may be mounted on the swingarm, to allow hands-free, stable positioning and orientation of imaging device 118 for desired image acquisition. In other embodiments, system 100 may comprise a portable, hand-held colposcope, which may be implemented in a common mobile device such as a cell phone or a smartphone.

System 100 described herein is only an exemplary embodiment of the present system, and may have more or fewer components than shown, may combine two or more components, or a may have a different configuration or arrangement of the components. The various components of system 100 may be implemented in hardware, software or a combination of both hardware and software, including one or more signal processing and/or application-specific integrated circuits. In various embodiments, system 100 may comprise a dedicated hardware device, or may form an addition to or extension of an existing medical device, such as a colposcope. In addition, aspects of the present system which can be implemented by computer program instructions, may be executed on a general- purpose computer, a special-purpose computer, or other programmable data processing apparatus.

### Automated Monitoring and Guidance in Medical Imaging Procedures

For background and additional information regarding image analysis of cervical images, see, e.g., Wenjing Li, Wenjing Li, Jia Gu, Jia Gu, Daron Ferris, Daron Ferris, Allen Poirson, Allen Poirson, “Automated image analysis of uterine cervical images”, Proc. SPIE 6514, Medical Imaging 2007: Computer-Aided Diagnosis, 65142P (30 Mar. 2007); Shiri Gordon, Gali Zimmerman, Rodney Long, Sameer Antani, Jose Jeronimo, Hayit Greenspan, “Content analysis of uterine cervix images: initial steps toward content based indexing and retrieval of cervigrams”, Proc. SPIE 6144, Medical Imaging 2006: Image Processing, 61444U (15 Mar. 2006); K. Fernandes, J. S. Cardoso and J. Fernandes, “Automated Methods for the Decision Support of Cervical Cancer Screening Using Digital Colposcopies,” in IEEE Access, vol. 6, pp. 33910-33927, 2018; Lange, Holger. “Automatic detection of multi-level acetowhite regions in RGB color images of the uterine cervix.” Medical Imaging 2005: Image Processing. Vol. 5747. International Society for Optics and Photonics, 2005; and Huang, Xiaolei, et al. “Tissue classification using cluster features for lesion detection in digital cervigrams.” Medical Imaging 2008: Image Processing. Vol. 6914. International Society for Optics and Photonics, 2008.

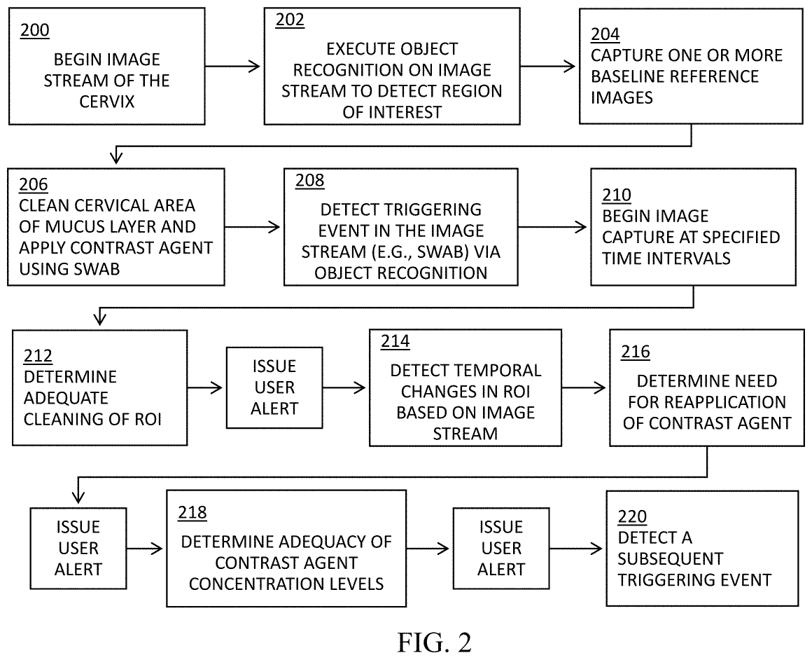

Reference is now made to FIG. 2, which is a flowchart illustrating an exemplary method according to certain embodiments of the present disclosure. The steps of the method are described herein with reference to the medical diagnostic procedure of colposcopy. As noted above, colposcopy involves the examination of an illuminated view of vaginal and cervical tissue, for the purpose of detecting and assessing premalignant and malignant lesions in these areas.

Initially, imaging device 118 of system 100 in FIG. 1 is positioned so as to gain a view of the cervix, e.g., through the opening of a speculum or similar device providing an internal view of the vaginal wall and the cervix. In some variations, such speculum or similar device may be attached to imaging device 118. In other variations, a speculum-like extension may be integrally formed with imaging device 118. Alternatively, imaging device 118 or a forward portion thereof may be received and guided within an opening of a speculum, e.g., through guide means.

In some embodiments, select parameters of imaging device 118 are adjusted, including, but not limited to, white balance, exposure time, gain (ISO), and/or gamma. In some embodiments, the images from imaging device 118 can be corrected by processing to adjust variations in any of these parameters. Illumination parameters (not associated with imaging device 118) can also be adjusted and recorded by system 100. Calibration images can be acquired from known targets (e.g., reflectance targets, color targets, resolution targets, distortion targets), and used to correct raw images. The advantage of adjusting these parameters is to be able to detect in the cervix whether or not there are external light sources in the images. External light sources that are not accounted for can potentially pose problems for algorithms that run on calibrated data that assumed certain tissue imaging conditions.

At a step 200, imaging device 118 begins the acquisition of images of an area of the cervix under observation. The images acquired by imaging device 118 may be a sequence of still images or a continuous video stream. In some instances, one or more baselines image of the cervix is captured at this stage, to use as base reference to assess any changes as a result of the aceto-whitening. In some embodiments, the images acquired by imaging device 118 are displayed live on display 116a. The images acquired by imaging device 118 may also be magnified or otherwise manipulated, such as by applying to the images colored filters, polarization, glare removal, and/or other techniques for improved visualization.

### Automated Identification of Cervical ROI

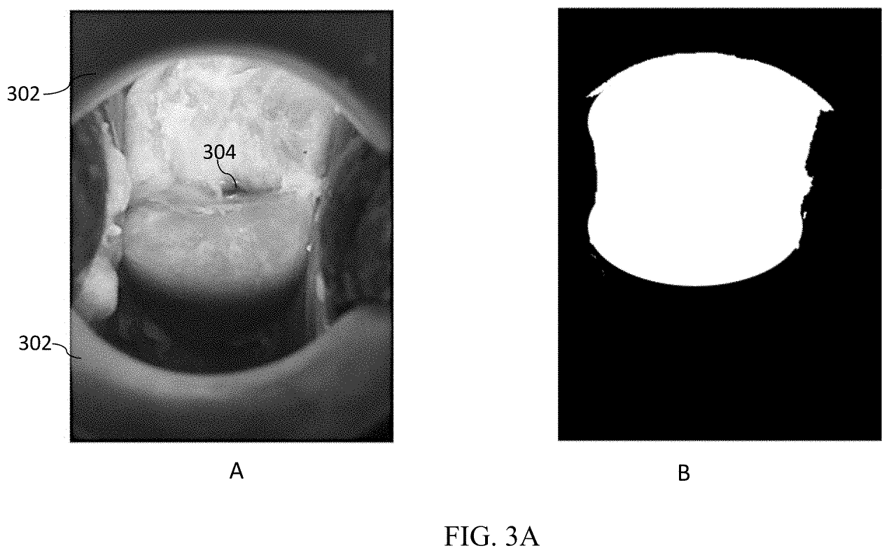

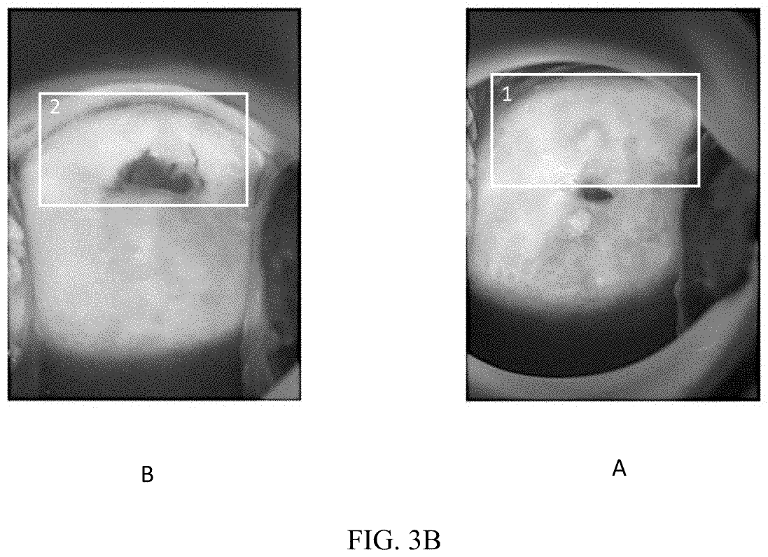

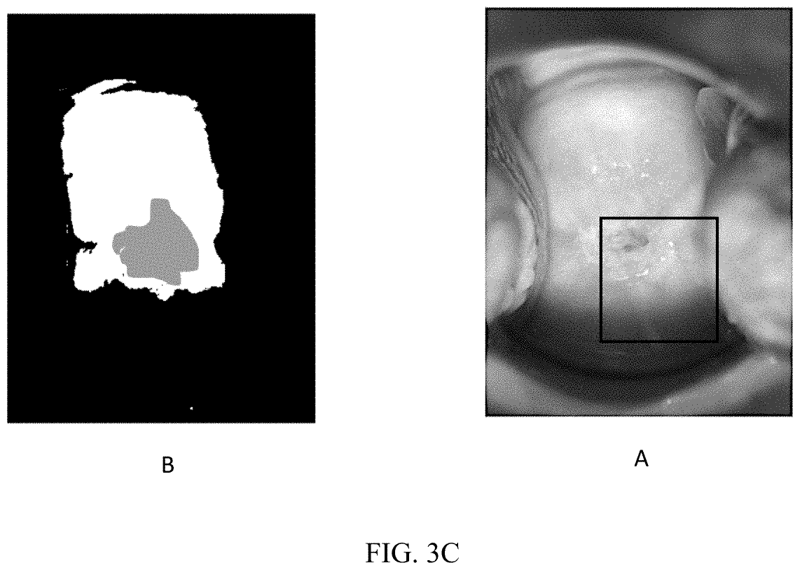

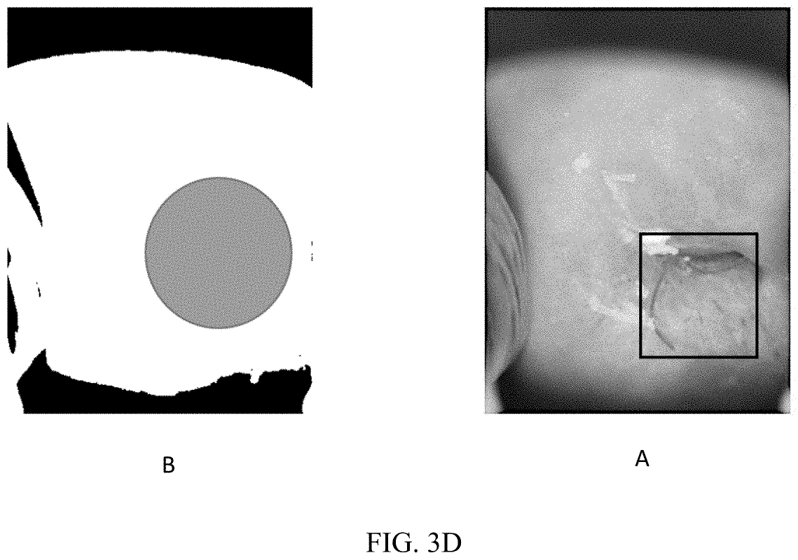

At a step 202, image processing module 110a applies continuous visual recognition to the images streamed by imaging device 118, identify and delineate the boundaries of the cervix in the image stream. FIG. 3A shows, in panel A, a reference image of a cervix captured through an opening of a speculum at a pre-cleaning stage.

In some embodiments, system 100 is then configured to identify and delineate the of a region of interest (ROI) comprising the boundaries of the cervix in the image stream. In some embodiments, the ROI detection may further comprise identifying one or more of the transformation zone (TZ), the cervical os, the squamo-columnar junction (SCJ), and/or the speculum.

For example, system 100 may be configured for identifying the TZ within the cervical area, which is the main ROI for purposes of detecting pathological tissue. The TZ is a region of the cervix where the columnar epithelium has been replaced and/or is being replaced by new metaplastic squamous epithelium. The TZ corresponds to the area of cervix bound by the original squamo-columnar junction (SCJ) at the distal end, and proximally by the furthest extent that squamous metaplasia has occurred as defined by the new squamo-columnar junction. In premenopausal women, the TZ is fully located on the ectocervix, and may move radially back and forth over the course of the menstrual cycle. After menopause and through old age, the cervix shrinks with the decreasing levels of estrogen. Consequently, the TZ may move partially, and later fully, into the cervical canal. Identifying the transformation zone is of great importance in colposcopy, as almost all manifestations of cervical carcinogenesis occur in this zone.

In some embodiments, system 100 may further be configured for identifying, e.g., incorrectly positioned speculum portions which may at least partially occlude the TZ in the image. Following this, system 100 may be configured for issuing an appropriate alert to the clinician performing the procedure to reposition the speculum. As noted above, the alert to the clinician may be, e.g., a visual, auditory, and/or verbal alert, communicated through display 116a and/or speaker 116c.

In some embodiments, system 100 may be further configured for identifying vaginal walls in the cervical image, when, e.g., portions of laxed and/or prolapsed vaginal wall may visually obstruct at least a portion of the TZ. Accordingly, if system 100 determines that a gap or discontinuity exists within the TZ mask due to obstruction by the vaginal walls, system 100 may be configured for issuing an appropriate alert to the clinician performing the procedure. In some embodiments, such an alert may include instructions to further open the vaginal walls, e.g., using an appropriate medical accessory. In some cases, lax or prolapsed vaginal walls may be pushed aside using, e.g., a vaginal wall retractor. In other cases, the clinician may slide a condom (with the condom tip removed) over the blades of the speculum. Following the issuance of the alert, system 100 may be configured for re-evaluating the positioning of the vaginal walls, before issuing an appropriate indication to the clinician to proceed with, e.g., a diagnostic and/or therapeutic procedure.

In some embodiments, system 100 may be configured for identifying and delineating the cervix in the pre-cleaning reference image, thus creating a cervix mask (e.g., white area in panel B in FIG. 3A). For example, system 100 may apply a specified color filter, such as a filter which selects for pink/red/white, for color-based identification of the cervix, based on normal cervical tissue color. In some embodiments system 100 may use such techniques as color augmentation to highlight visible changes in tissue.

In some embodiments, image processing module 110a may apply a hue, saturation, value (HSV) mask to find in the images candidates which could be the cervix, using morphological operations to fill in “holes” in the found cervix. The Kernels are defined as fraction of the image size (which may also account for digital zoom in the images). The threshold values here are set manually, and can be adjusted by using K-means and/or decision tree. A sample code for this operation may be:

img = cv2.cvtColor(img,cv2.COLOR_BGR2HSV)

kernelSize=11 lim=[[120, 200],[30, 255],[0, 256]]
cervixMap = np.uint8(np.where(((img[:,:,0]>=lim[0][0]) *

(img[:,:,0]<=lim[0][1])) *\

((img[:,:,1]>=lim[1][0]) * (img[:,:,1]<=lim[1][1])) *\
((img[:,:,2]>=lim[2][0]) *

(img[:,:,2]<=lim[2][1])),img[:,:,2],0))

cervixMap = cv2.morphologyEx(cervixMap, cv2.MORPH_OPEN,
kernelOpen)
cervixMap = cv2.morphologyEx(cervixMap, cv2.MORPH_CLOSE,
kernelClose)
img[:,:,2] = np.uint8(np.where(cervixMap>0,img[:,:,2],0))

In some embodiments, if the cervix is poorly oriented in the images, system 100 may be configured for issuing an alert to the clinician performing the examination toto re-position the imaging device 118. Similarly, if noticeable shading is detected, or the area is too large or too small, a suitable alert may issue prompting the clinician, e.g., to change a height, orientation, distance, and/or angle, and/or remove an obstruction which may be causing the shadow.

In other embodiments, system 100 may employ one or more known computer vision methods for identifying the cervix and segmenting it from, e.g., the visible parts of the speculum 302 and the cervical os 304. For example, image processing module 110a may employ a dedicated object recognition and classification application for identifying one or more regions, features, and/or objects in the image. The application may first conduct feature-level detection, to detect and localize the objects in the image, and then perform decision-level classification, e.g., assign classifications to the detected features, based on the training of the application. The application may use machine learning algorithms to train it to identify objects in given categories and subcategories. For example, the application may use one or more machine learning algorithms, such as a support vector machine (SVM) model and/or a convolutional neural network (CNN). SVMs are supervised learning models that analyze data used for classification and regression analysis. Given a set of training examples, each marked as belonging to one or the other of two categories, an SVM training algorithm builds a model that assigns new examples to one category or the other, making it a non-probabilistic binary linear classifier. An SVM model is a representation of the examples as points in space, mapped so that the examples of the separate categories are divided by a clear gap that is as wide as possible. New examples are then mapped into that same space and predicted to belong to a category based on which side of the gap they fall.

CNNs are a class of deep, feed-forward artificial neural networks, most commonly applied to analyzing visual imagery. A CNN algorithm may be trained by uploading multiple images of items in the category and subcategories of interest. For example, the CNN algorithm may be trained on a training set of properly-labeled relevant images, which may include multiple cervical images of different sizes, taken from different angles and orientations, under varying lighting conditions, and with different forms of obstruction and interferences. The CNN algorithm may also be provided with a set of negative examples, to further hone the classifier training. The CNN algorithm applies a convolutional process to classify the objects in each training image in an iterative process, in which in each iteration, (i) a classification error is calculated based on the results, and (ii) the parameters and weights of the various filters used by the CNN algorithm are adjusted for the next iteration, until the calculated error is minimized. This means that the CNN algorithm can be optimized to recognize and correctly classify images from the labelled training set. After training completes, when a new image is uploaded to the CNN algorithm, the CNN algorithm applies the same process using the parameters and weights which have been optimized for the relevant category, to classify the new image (i.e., assign the correct labels to objects identified therein) with a corresponding confidence score.

### Automated Area Preparation Monitoring

In some embodiments, at a step 204, system 100 may be configured to capture one or more baseline reference images of the cervix at a pre-cleaning stage.

In some embodiments, at a step 206, a clinician may then proceed to perform a cleaning stage of the colposcopy, in which excess mucus is removed from the cervix, e.g., by applying a dry or saline-soaked swab. System 100 may then be configured for capturing one or more post-cleaning images or image frames, to record a post-cleaning reference image of the cervix, for later comparison after application of the contrast agent.

In some embodiments, system 100 is configured for detecting incomplete removal of a mucosal layer of the cervix during the preparation stage of a colposcopic examination. Colposcopy typically starts with the insertion of a speculum and the positioning of a colposcopic imaging device, such as imaging device 118, for visualization of the cervix through the speculum. System 100 may then be configured to capture an initial reference image and/or begin a continuous video stream of the cervix.

In some embodiments system 100 may be configured for detecting and delineating, in the post-cleaning reference images, areas of the cervix which already are visibly white, e.g., area exhibiting leukoplakia (hyperkeratosis). For example, with reference to FIG. 3B, a leukoplakia area is marked by rectangle 1 in Panel A, pre-acetowhitening, and in rectangle 2 in panel B, post-acetowhitening. Such areas may be marked for exclusion from further analysis based on the effects of aceto-whitening.

At a later step 212, with reference to FIG. 3C, system 100 may then be configured for comparing the pre- and post-cleaning images to each another, to detect areas which have not been thoroughly and/or consistently cleaned of mucus. For example, panel A in FIG. 3C shows mucus covering the columnar epithelium area of the cervical canal. Accordingly, image processing module 110a may be configured for comparing an optical signal observed from the compared images, such as pixel value. Based on the comparison, system 100 may be configured to detect areas in which a difference in the observed optical signal is below a specified threshold. In some embodiments, image processing module 110a may create a mask of the non-cleaned areas (e.g., the rectangle in panel A, and the grey area in panel B). After, e.g., morphological dilation, if it is determined that the mask is above a certain size, then system 100 may be configured for issuing an alert to the clinician performing the examination to attend to the area identified by system 100. The alert to the clinician may be, e.g., a visual, auditory, and/or verbal alert, communicated through display 116a and/or speaker 116c. In some embodiments, such alert may comprise an indication as to the location, within an image, of the one or more areas identified as non-cleaned. For example, system 100 may be configured for displaying an outline around the detected area(s), displaying a box surrounding the detected area(s), displaying an arrow pointing to the detected area(s), displaying a zoomed-in view of the detected area(s), and/or displaying an indication of one or more values associated with the detected area(s).

With reference to FIG. 3D, in some embodiments, system 100 may be configured for detecting areas which have not been thoroughly cleaned of mucus in cases where a patient has an Intrauterine Device (IUD), which may be susceptible for retaining excess mucus. Accordingly, in some embodiments, an object recognition and classification application of image processing module 110a may be trained to recognize, e.g., a dark string connected to the IUD (marked by the rectangle in panel A) in the center of an image, using one or more image segmentation algorithms. If found, an IUD mask (grey area in panel B) is created, and dilated multiple times. System 100 may then be configured to issue an alert to the clinician regarding the presence of an IUD in the cervix, which may require additional mucus removal steps. System 100 may also be configured for performing an additional cleaning verification, by comparing a pixel value difference between the reference image and post-cleaning image within the IUD mask area, as compared to a pixel value difference outside the IUD mask area. If the pixel value difference inside the IUD mask area is lower than the difference outside the IUD mask area, system 100 may issue an alert to the clinician to perform additional cleaning steps around the IUD area.

### Automated Identification of Medical Applicator

With continued reference to FIG. 2, at a step 208, system 100 may be configured to identify the presence of a specified medical accessory (in the case of colposcopy, the medical accessory is a medical applicator, such as a cotton swab) in the field of view of imaging device 118. In some embodiments, such identification may be used as a triggering event for capturing a plurality of images at or around specified time points thereupon. In some embodiments, the plurality of images may be captured and/or stored from a continuous stream of video from imaging device 118.

As used herein, the term “medical accessory” refers broadly to any medical and/or surgical accessory, tool, and/or instrument used in connection with medical and/or surgical procedures. Examples of medical accessories include, but are not limited to, medical applicators, absorbent carriers, or swabs used for swabbing, daubing, or applying a substance to a bodily tissue; scopes and probes, such as endoscopes; graspers, clamps and/or retractors; mirrors; cutters, such as scalpels, scissors, drills, rasps, osteotomes, trocars, and the like; dilators and specula; suction tips and tubes; staplers and other sealing devices; needles; powered devices; and measurement devices, such as rulers and calipers.

In some embodiments, additional and/or other objects may be identified, including non-reflective colored medical devices and/or accessories to assist in image detection and segmentation (e.g., a green/blue speculum which can be easily detected and segmented in images).

Accordingly, in preparation for step 204, a clinician applies a contrast agent to the area under observation using a cotton swab or a similar medical applicator. For example, as noted above, in colposcopy, diluted acetic acid is applied to the observed tissue by a cotton swab, to create, through the phenomenon of aceto-whitening, a visual contrast between healthy tissue and certain growths and abnormalities. The visual effect of aceto-whitening typically may peak at a specified time after application, for example at 120 seconds, however, in other scenarios, the effect of contrast media or other diagnostic protocols may develop by several stages or intervals, e.g., at 30 seconds, 45 seconds, and 90 seconds after application. Accordingly, to be able to perform a time-resolved assessment of the effect of the contrast media, it is imperative to capture an image or series of images of the area under observation at, or around, specified time points during this process.

Accordingly, in step 208, image processing module 110a identifies a specified object in the image stream, which serves as a triggering event for capturing one or more images at and/or around specified time points. In the case of colposcopy, image processing module 110a is configured to detect the presence of a medical applicator (such as a cotton swab) in the image stream. The identification of a cotton swab in step 208 is used as an indication of the application of the contrast agent, thus triggering image capture at a plurality of specified time points thereupon.

In other embodiments, or in connection with other practical applications, image processing module 110a may be trained to recognize a variety of different objects or combinations of objects. For example, image processing module 110a may be trained to detect and recognize the simultaneous presence of more than one object in the field of view, e.g., a combination of two or more medical applicators, and/or a medical applicator and another medical accessory or device being used concurrently. In other variations, image processing module 110a may be trained to track a medical applicator and/or another object in the field of view, whereby the triggering event may be a specified distance from a reference point in the cervix; a specified position and/or orientation with respect to a reference point in the cervix; and/or a specified action, gesture, or motion involving the medical applicator. For example, image processing module 110a may be trained to detect a cotton swab making contact with a specified bodily area or tissue, a specific position or orientation of the cotton swab, the continued present of the swab for a specified period of time, and/or the removal of the swab from the bodily area, to trigger the image capture process. In other variations, the triggering event may be based on one or more of, e.g., human hand gesture recognition, facial recognition, textual recognition, and/or voice command. In all such cases, the detection by image processing module 110a of one or more objects, combinations thereof, or another item or event, may cause the system 100 to determine the occurrence of a triggering event.

In some embodiments, system 100 may be configured to differentiate between uses of a swab which are associated with the application of contrast agent, and other, related and/or supporting uses of a swab, which should not trigger the image capture process. For example, in the course of colposcopy, swabs may be used for one or more of washing the cervix in saline, cleaning up blood from trauma cause by inserting the speculum, applying Monsel's solution after taking a biopsy, applying local pressure after taking a biopsy, and/or repositioning the cervix and/or portions thereof All such uses should be differentiated from using the swab for the application of a contrast agent. Accordingly, in some embodiments, system 100 may be configured to determine a related and/or supporting use of a swab within the image stream, based on one or more of a location of the swab within the cervix area, a location of a contact point of the swab with the cervix, a duration of the presence of the swab within the cervix area, a timing of the appearance of the swab in the image stream, a color of a solution applied to the tip of the swab, and/or a specified size and/or shape of the swab.

In some variations, system 100 may comprise, e.g., a database stored on storage device 114, of preprogrammed triggering events associated with various procedures and practical applications, which may be available for user selection.

Image processing module 110a may employ a dedicated object recognition and classification application for identifying one or more triggering objects or events in the image stream.

In some embodiments, image processing module 110a may employ one or more known image recognition techniques. A sample code for this purpose may be:

swabMap = np.uint8(np.maximum(np.where(img[:,:,1]<5,img[:,:,2],0),
kernelSize=3lim=[[70,120],[0,100],[50,256],[0.4,3]] N=3

np.where(((img[:,:,0]>=lim[0][0]) * (img[:,:,0]<=lim[0][1])) *\

((img[:,:,1]>=lim[1][0]) * (img[:,:,1]<=lim[1][1])) *\
((img[:,:,2]>=lim[2][0]) * (img[:,:,2]<=lim[2][1])),\
img[:,:,2],0)))

#Remove small speckels
swabMap = cv2.morphologyEx(swabMap, cv2.MORPH_OPEN,
kernelOpen)
#Fill holes
swabMap = cv2.morphologyEx(swabMap, cv2.MORPH_CLOSE,
kernelClose)
#Remove swab - thin lines
swabMap = cv2.morphologyEx(swabMap, cv2.MORPH_OPEN,
kernelOpen1D)
swabMap = cv2.morphologyEx(swabMap, cv2.MORPH_OPEN, \

np.transpose(kernelOpen1D))

Once the morphological operations are done, the images histogram can be used to verify that the proposed ROI is part of the brightest area in the image. This assumes that the reflectivity of the cotton swab is >0.9 and that of the cervix is <0.6:

#Use histogram to match the detected oprtions and the image
imgHist, bins = np.histogram(img[:,:,2],bins=255)
swabHist, bins = np.histogram(swabMap,bins=255)
ratioMean = np.convolve(np.true_divide(imgHist[1:],swabHist[1:]),\

np.ones((N,))/N, mode=‘valid’)

minValVal = [setBin for ratioVal,setBin in zip(ratioMean,bins[2:−2]) \

if lim[3][1]>ratioVal>lim[3][0] ]

if len(minValVal)>0:

minValVal = minValVal[0]

else:

minValVal = 255
#Apply swabMap mask
img[:,:,2] = np.uint8(np.where(swabMap>0,img[:,:,2],0))
#Apply minimal value value
img[:,:,2] = np.uint8(np.where(img[:,:,2]>minValVal,img[:,:,2],0))

The application may first conduct feature-level detection, to detect and localize the object in the image, and then perform decision-level classification, e.g., assign classifications to the detected features, based on the training of the application. The application may use machine learning algorithms to train it to identify objects in given categories and subcategories. For example, the application may use a convolutional neural network (CNN), which is a type of neural network that has successfully been applied to analyzing visual imagery. A CNN algorithm, in the convolutional step, first applies various filters to extract multiple feature maps of the image, wherein each filter has a set of parameters and is assigned a weighting. A pooling step may then be applied to provide subsampling of the convolved feature maps and reduce the amount of data. The convolutional and pooling steps may then be repeated on the results of the first iteration, to create multiple layers. The CNN algorithm may then apply a multi-layer perception to the multiple layers, to create a fully-connected layer, which is used to classify the detected features of the object in the image against the categories on which the CNN algorithm was trained. As an output, the CNN algorithm may assign probabilities of between 0 and 1 for the object against each category.

Training of the CNN algorithm may be conducted by uploading multiple images of items in the category and subcategories of interest. For example, the CNN algorithm may be trained on the category of common medical accessories, which may include a subcategory of medical applicators. The CNN algorithm is provided with relevant images appropriately labelled, which may include multiple images of cotton swabs and similar applicators of different shapes, types, and sizes; as disposed in various positions and orientations; and against varying backgrounds relevant to the task at hand. In the present context, the CNN algorithm may be provided with images of medical applicators against a background of cervical or similar tissue, to train the CNN algorithm on practical, real-world situations which may be encountered in runtime. The CNN algorithm may also be provided with negative examples, of images that are not medical applicators, to further hone the classifier training. Negative examples for the classifier ‘cotton swabs’ may include, e.g., images of medical accessories in other subcategories, such as tongue depressors, scissors, tweezers, and/or clamps. The CNN algorithm applies the convolutional process described above, and classifies the object in each training image in an iterative process, in which in each iteration, (i) a classification error is calculated based on the results, and (ii) the parameters and weights of the various filters used by the CNN algorithm are adjusted for the next iteration, until the calculated error is minimized. This means that the CNN algorithm can be optimized to recognize and correctly classify images from the labelled training set. After training completes, when a new image is uploaded to the CNN algorithm, the CNN algorithm applies the same process using the parameters and weights which have been optimized for the relevant category, to classify the new image (i.e., assign the correct labels to objects identified therein) with a corresponding confidence score. It will be appreciated that, in runtime, the CNN algorithm will need to identify such objects in an unconstrained manner, i.e., at varying angles with respect to the imaging device, under different lighting conditions, and/or partially occluded, etc. Therefore, to ensure a high level of confidence in the results of the CNN algorithm, certain training guidelines and conditions need to be met. For example, the images need to be of at least a certain quality and resolution. A minimum number of training images per subcategory is recommended. Advantageously, an equal number of positive and negative training images, as well as images with a variety of setting and backgrounds, should be used.

### Automated Image Capture Based on Triggering Events

With continued reference to FIG. 2, at a step 208, image processing module 110a detects one or more a triggering events, such as the application of acetic acid with a cotton swab, in the image stream, to indicate the beginning of, e.g., the acetowhitening process. As noted above, in some embodiments, the triggering event may be defined as the presence of the swab for a specified period of time, or the removal of the swab within or after a specified period of time. In some embodiments, as shall be further detailed below, two or more triggering events may occur in succession, e.g., upon reapplication of acetic acid in case of improper first application and/or inadequate acid concentration. In some embodiments, system 100 may be configured for differentiating specified uses of a swab as triggering events from other uses which are not deemed triggering events, based on a plurality of determining factors.

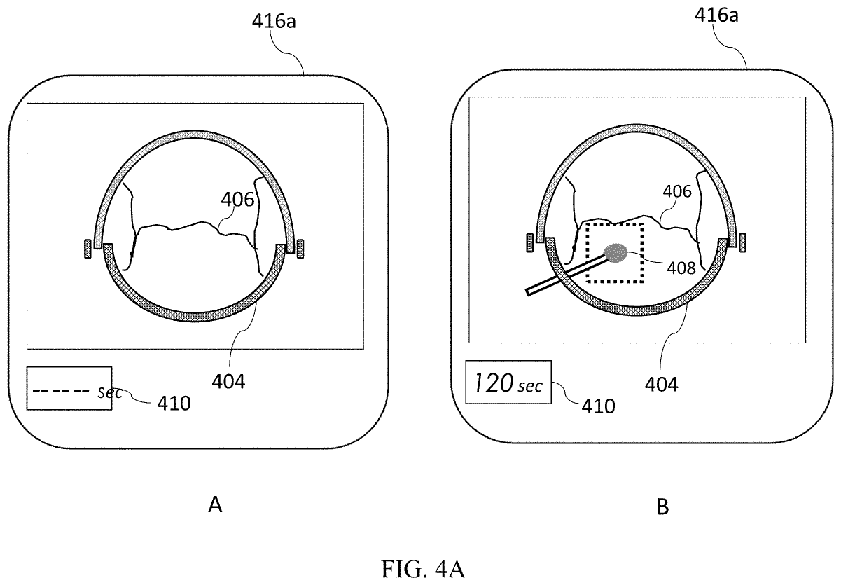

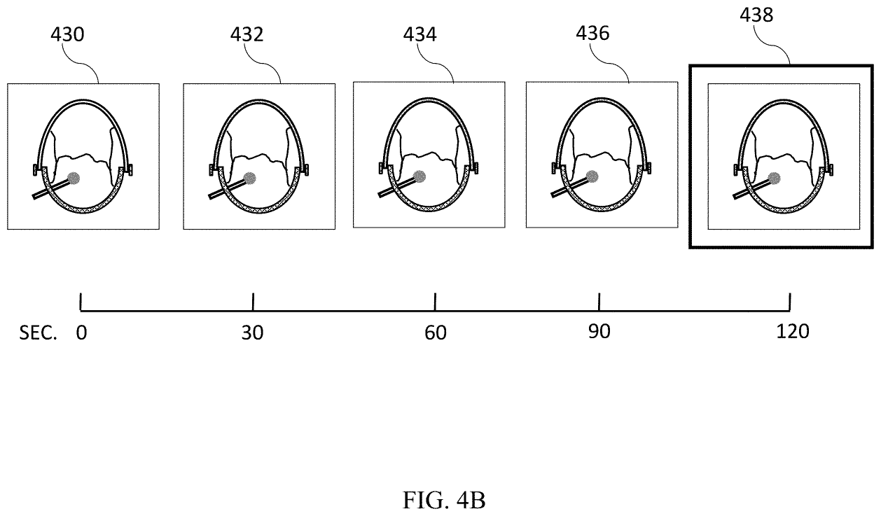

FIG. 4A schematically illustrates an exemplary application of system 100 of FIG. 1 in a colposcopy procedure. In panel A, a display monitor, such as display 116a, shows images acquired by imaging device 118 of a cervical area 406 through an opening of a speculum 404. In panel B, a cotton swab 408 is inserted into the cervix to apply a contrast agent, such as acetic acid, to cervix area 406. Image processing module 110a identifies cotton swab 408 in the image stream (indicated by dashed rectangle around the tip of swab 408 ), thereby triggering a countdown of, e.g., 120 seconds, which may be displayed in real time on display 116a via countdown timer 410.

With reference back to FIG. 2, upon the occurrence of a triggering event in step 208, system 100 may be configured to capture, at a step 210, one or more images of the area under observation at, or around, specified time points. The term “upon”, as used herein, may refer to the capture of one or more images immediately when the triggering event is identified, and/or to the capture of one or more images after the triggering event has been identified. For example, in the case of colposcopy, system 100 may capture one or more images immediately upon the occurrence of the triggering event and at various time points thereafter (e.g., 30, 45, 60, 90, and/or 120 seconds). As indicated by the exemplary timeline in FIG. 4B, imaging device 118 may stream a series of images 430-438. Upon detection of a cotton swab in image 430, system 100 may capture a series of images, e.g., images 430, 432, 434, 436, at time intervals 0, 30, 60, 90, and 120 seconds, respectively. Optionally, system 100 may acquire a continuous or a time-lapse video of the entire process. Some embodiments of the present invention involve capturing a sequential or periodic series of images according to predetermined protocols; capturing a sequence (or burst) of images around specified time points (e.g., slightly before and/or after); or capturing multiple sequential images and selecting one or more images from the sequence based on an image quality parameter. In the context of a continuous stream of images or a video acquired by imaging device 118, processed by system 100 and displayed of display 116a, capturing an individual image at a specific time point may involve, e.g., capturing and saving one or more image frames corresponding to specific time points, and/or applying suitable captioning or annotation to one or more image frames in a video, representing a snapshot of the image stream at the desired time point. The captured image(s) may then be stored in an image database on storage device 114. For example, system 100 may be configured to mark or annotate stored image with necessary information or metadata, such as patient or user name, date, location, and other desired information. In some embodiments, patient metadata may include information regarding specified procedure and/or assistance measures needed in the course of image capture, to ensure future consistent results. In some embodiments, system 100 may generate automatic electronic patient records comprising all images captured over multiple procedures in connection with a patient or user, to enable, e.g., post-exam reviews, longitudinal tracking of changes over time, and the like. Such records may be stored in a database on storage device 114. In some variations, patient records may be EMR (electronic medical records) compatible, to facilitate identifying patients who are due for preventive visits and screenings, and monitor how patients measure up to certain parameters. EMR records may then be shared over a network through, e.g., communications module 114.

In some embodiments, one or more countdowns may be adjusted to account for, e.g., contrast agent type, applicable medical procedures practiced by a medical organization, and/or ambient conditions (e.g., temperature, relative humidity) which may necessitate different countdowns of various durations may be run sequentially or in parallel.

### Automated Analysis of Contrast Agent Effect

In step 210 In FIG. 2, system 100 may be configured for capturing an image stream, and/or a plurality of images in specified successive time instances, and/or a continuous video stream, of the cervix, before and after the triggering event discussed above, to document both (i) every spatial point of the tissue area under analysis, and (ii) temporal progression of the alterations in the tissue over time.

Then at a step 214, system 100 may then be configured for capturing and processing a temporal succession of images of the tissue under observation. Such processing may involve measuring optical properties of the light that is reemitted from the tissue under observation, including in the form of reflection, diffuse scattering, fluorescence, or any combinations thereof. Individual image frames so captured may be stored successively, e.g., on storage device 114. System 100 may then be configured for calculating quantitative parameters of these measured optical properties as a function of time in every spatial point of the area under analysis, which allows for an evaluation of the intensity and extent of the provoked acetowhitening. For example, system 100 may calculate one or more acetowhitening response curves for acetowhitening intensity, based on these measurements. The response curves may then be compared to known response curves associated with various types of tissue pathological classifications. The quantitative parameters may include at least some of: the maximum value of acetowhitening recorded; the time period required for reaching the maximum response value; the total ‘volume’ of acetowhitening response during a specified time period (e.g., the integral or area under a graph representing the response curve); the rate of intensity increase to maximum value; and the rate of intensity decrease starting from the maximum value of the response curve. In some embodiments, such derived parameters may be further weighed, e.g., based upon features particular to the tissue sample examined (such as patient age) and/or features characterizing the regional population of the subject whose tissue is examined.

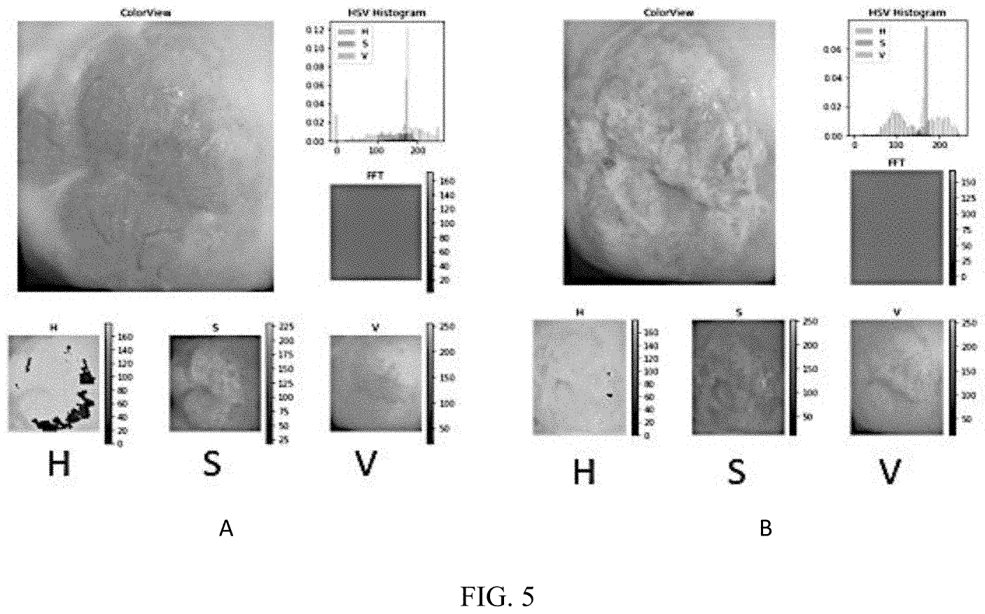

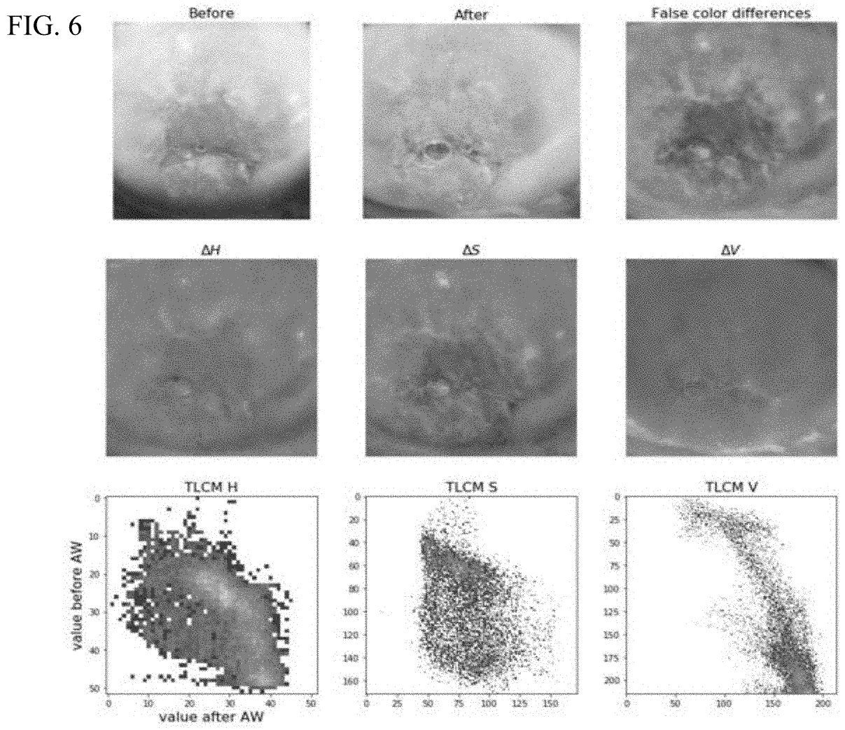

As shown in FIG. 6, in some embodiments, the image stream may be converted into the hue, saturation, value (HSV) or the La*b* color spaces. In the HSV color space, each component shows the color, chromaticity and lightness. Thus, domain knowledge can be used to measure the amount of change in each pixel and each color channel. The variation in color channels can be shown by plotting the value of a pixel after the application of the contrast agent, as a function of its value before. In some embodiments, statistical calculation designed for use with a gray-level co-occurrence matrix (GLCM) may be used. The values may then be fed into a decision tree or a classifier. FIG. 5 demonstrates an exemplary analysis of pre- (panel A) and post-acetowhitening (panel B) images in the HSV color space. In some embodiments system 100 may use such techniques as color augmentation to highlight visible changes in tissue.

In some embodiments, system 100 may be configured for tracking changes overtime based, at least in part, on, HSV/L*ab/RGB changes; spatial features, such as sharp edges; identification of external objects based on changes in sequence of images (e.g., by subtracting consecutive images); and/or using optical flow to identify objects moving in or out of image frame, using, e.g., single shot detection (SSD).

In this regard, it should be noted that the time-resolved effect in aceto-whitened tissue may vary depending on the classification of the tissue in question. For example, a categorization used commonly in clinical practice regards HPV infection, tissue inflammation, CIN 1, or a combination thereof, as low-grade squamous intraepithelial lesions (LSIL). Conversely, high-grade squamous intraepithelial lesions (HSIL) include CIN 2, CIN 3, and invasive carcinoma. In this regard, it has been observed that, e.g., HPV-classified tissue presents an acetowhitening time-resolved response which increases rapidly before reaching a relatively stable saturation level. The acetowhitening response curve corresponding to inflammation reach a higher peak value earlier than HPV-classified tissue, but then decay relatively, abruptly. In CIN 1-classified tissue, the acetowhitening response curves reach their maximum later than the curves corresponding to HPV or inflammation, and then decay with a rate that is notably slower than that observed in the inflammation cases. Finally, for tissues classified with HSIL, the maximum acetowhitening response of the curves is reached later and with a higher value than that observed in the HPV and CIN 1 cases; however, the decay rate for HSIL lesions much slower than that seen in the inflammation-classified curves.

In some embodiments, the response curves calculated for the acetowhitening effect during the examination may be further employed to detect the need for one or more re-applications of a contrast agent and/or the adequacy of the concentration of the contrast agent being used. In this regard, as noted above, LSIL tissue, immature squamous metaplasia, and inflammatory lesions typically require prolonged evaluation times to reach a correct conclusion. Accordingly, in some cases, multiple applications of contrast agent may be required to prolong the acetowhitening effect and, thus, the effective observation window. In other embodiments, system 100 may be configured for determining whether the concentration levels of the contrast agent are adequate.

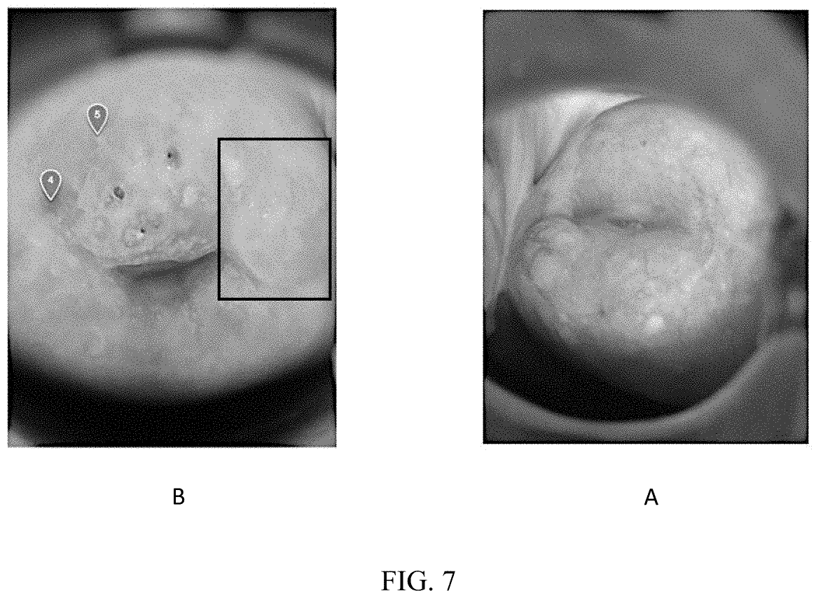

With reference to FIG. 7, as marked by the rectangle in panel A, a cervical area has undergone an acetowhitening response, which may correspond to a tissue pathology. Panel B shows a cervical tissue where the effect of acetowhitening has reversed. By calculating the timing and rate of acetowhitening intensity decay in the tissue under observation, system 100 may be configured for determining, e.g., the type of pathology represented by the measured optical properties of the tissue, and, in turn, whether additional applications of contrast agent are warranted. For example, if system 100 determines that the acetowhitening intensity decay curve corresponds to that of LSIL tissue, system 100 may indicate for additional one or more applications of contrast agent, to prolong the acetowhitening effect and, thus, the effective observation window.

In some embodiments, at a step 216, system 100 may be configured for issuing an appropriate alert to the clinician to reapply the contrast agent to the cervical region. In some embodiments, system 100 is further configured for monitoring the field of view of imaging device 118, until automatically detecting a reapplication of the contrast agent, by, e.g., detecting a swab as described above. In such embodiments, system 100 may further be configured to issue a successive series of escalating alerts if such swab detection does not occur within a specified period of time, which may be between 10-45 seconds. Such escalating alerts may include an increase in a visual alert on display 116c and/or an increase in a volume of an auditory and/or verbal alert sounded by speaker 116c.

Upon a reapplication of contrast agent, system 100 may be configured for further continuously measuring the optical properties of the light that is reemitted from the tissue under observation, and calculating the quantitative parameters of these measured optical properties as a function of time. In some embodiments, system 100 may be configured for repeating one or more times the steps of detecting acetowhitening decay, automatically monitoring for the reapplication of the contrast agent, and issuing one or more appropriate alerts, as described above. For example, system 100 may be configured for repeating these steps a total number of between 2 and 6 times.

In some embodiments, the need for reapplication of a contrast agent may be determined, based, at least in part, on ambient parameters, such as temperature, relative humidity, etc. Such parameters may be acquired by sensors within system 100 itself and/or from outside sources.

In some embodiments, at a step 218, system 100 may further be configured to take into account other observed phenomena and/or pathologies in the tissue, to determine whether a particular set of acetowhitening decay measurements may be the result of inadequate contrast agent concentration levels. For example, as noted above, a relatively slow time-resolved decay rate of the acetowhitening effect may indicate the presence of a HSIL-categorized tissue. However, if other, earlier indications taken during the examination do not support such conclusion, the slower signal decay rate may be an indication of too low contrast agent concentration (e.g., 3% acetic acid instead of 4-5%). Accordingly, if an initial review of the tissue, e.g., under green light as described above, does not reveal atypical vascularity, then a conclusion may be drawn that the cause of the slow rate of reversal is lower contrast agent concentration. Accordingly, system 100 may issue an appropriate alert to the clinician to check the contrast agent concentration level.

In some embodiments, system 100 may be configured for making these determination across multiple successive examination of a plurality of patients. For example, if a similar decay rate pattern emerges in several successive examinations, system 100 may be configured for determining that the culprit is more likely to be lower contrast agent concentration levels.

In some embodiments, system 100 may be further configured for monitoring the time-resolved effects of the application of Lugol's iodine as the contrast agent. The principle behind the iodine test is that original and newly formed mature squamous metaplastic epithelium is glycogenated, whereas CIN and invasive carcinoma contain little or no glycogen. Because iodine is glycophilic, the application of an iodine solution results in uptake of iodine in glycogen-containing epithelium. Therefore, the normal glycogen-containing squamous epithelium stains mahogany brown or black after application of iodine. Columnar epithelium does not take up iodine and remains unstained, but may look slightly discolored due to a thin film of iodine solution. Areas of immature squamous metaplastic epithelium may remain unstained with iodine or may be only partially stained. If there is shedding (or erosion) of superficial and intermediate cell layers associated with inflammatory conditions of the squamous epithelium, these areas do not stain with iodine and remain distinctly colorless in a surrounding black or brown background. Areas of CIN and invasive carcinoma do not take up iodine (as they lack glycogen) and appear as thick mustard yellow or saffron-colored areas. Areas with leukoplakia do not stain with iodine. Condyloma may not, or occasionally may only partially, stain with iodine. Accordingly, the use of Lugol's iodine in colposcopic practice may form a supplementary step which helps in identifying lesions overlooked during examination with saline and acetic acid, as well as delineating the anatomical extent of abnormal areas more clearly, thereby facilitating treatment.

### Use of Machine Learning Algorithms

In some embodiments, the present invention may apply one or more techniques based in artificial intelligence (AI), machine learning, and/or convolutional neural networks for analyzing images acquired in the course of a medical imaging procedure, such as colposcopy.

For example, instead of adjusting technical parameters of imaging device 188, as described above, an algorithm of the present invention may be configured to apply, e.g., a trained classifier to classify images based on specified quality parameters. Such classification can be applied by, e.g., image processing module 110a of system 100 to assess whether an image meets specified standards (e.g., sharpness, location of cervix in image, etc.) for being used in image-based diagnosis, and to issue a suitable alert instructing a user of system 100 on ways to improve image acquisition.

In some embodiments, such classifier may be trained on a large number of images, e.g., between 5,000 and 25,000, wherein the training set is manually labelled with image quality annotations (e.g., on an ordinal scale of bad, fair, good and excellent). In some embodiments, parameters for judging quality of images may include, but are not limited to, image focus, lack of artifacts (e.g., mucous/discharge, blood, swap, etc.), observability of four quadrants of the cervix, lack of shadows occluding the cervix, proper modality (e.g., selecting for green color filter and/or Lugol's iodine test), etc. The annotations may be provided by experts, such as colposcopists and image analysis experts. Such a classifier can be used as a subroutine in some of these algorithms.

In some embodiments, a classifier of the present invention may use features extracted from a Convolutional Neural Network (CNN), as well as manually-annotated features identified as relevant for the analysis of blind image quality assessment (e.g., BRISQUE descriptor). The final quality results from a linear aggregation of the aforementioned semantic features with positive weights constraints (see, e.g., Fernandes, Kelwin, Jaime S. Cardoso, and Jessica Fernandes. “Transfer learning with partial observability applied to cervical cancer screening.” Iberian conference on pattern recognition and image analysis. Springer, Cham, 2017; Silva, Wilson, et al. “Towards Complementary Explanations Using Deep Neural Networks.” Understanding and Interpreting Machine Learning in Medical Image Computing Applications. Springer, Cham, 2018. 133-140; Mittal, Anish, Anush Krishna Moorthy, and Alan Conrad Bovik. “No-reference image quality assessment in the spatial domain.” IEEE Transactions on Image Processing 21.12(2012): 4695-4708). Thus, by supervising the latent activations of the deep neural network, it is possible to instruct a user of the system on how to correct the acquisition.

Additional image quality classifiers which may be used in the framework of present embodiments are those discussed in Fernandes, Kelwin, Jaime S. Cardoso, and Jessica Fernandes. “Transfer learning with partial observability applied to cervical cancer screening.” Iberian conference on pattern recognition and image analysis. Springer, Cham, 2017; and Mittal, Anish, Anush Krishna Moorthy, and Alan Conrad Bovik. “No-reference image quality assessment in the spatial domain.” IEEE Transactions on Image Processing 21.12(2012): 4695-4708.

In some embodiments, an image quality classifier of the present invention may be configured to select relevant images from the image stream and/or image sequence, be selecting for image quality based, at least in part, on the parameters described above.

In some embodiments, image processing module 110a may further be configured to be applied in one or more of the previous stages described above, including, ROI identification; cropping the cervix; identification of additional anatomical ROIs (e.g., the cervical os, the speculum, the TZ, SCJ, and/or vaginal walls); identification of swab to initiate timer countdown, etc.

Once all features in the images have been identified, a machine learning algorithm of the present invention may be applied to identify, e.g., pre- and post-acetowhitening images, to assess if acetic acid was properly applied. For example, such an algorithm may comprise a pairwise model configured to makes a prediction based on receiving two modified images and concatenating their latent representations. In some embodiments, such algorithm may be configured to compare all pre- to all post-acetowhitening images; only a first and last image in a sequence; images captured at the beginning and end of a specified period of time; and/or pre- and post-images meeting a specified quality threshold.

In some embodiments, an algorithm of the present invention may be configured to automatically determine proper application of acetic acid. For example, the algorithm may apply a pairwise Siamese model, wherein the algorithm receives two cropped images of the cervix (potentially registered and aligned), and outputs the probability of acetic acid having been applied to the ROI. In some embodiments, the algorithm may use a segmentation network (e.g., U-Net) to detect regions with external artifacts (e.g., blood, mucous) and regions with missing acetic acid. System 100 may then identify these regions to a user of the system. (See, e.g., Fernandes, Kelwin, Jaime S. Cardoso, and Birgitte Schmidt Astrup. “A deep learning approach for the forensic evaluation of sexual assault.” Pattern Analysis and Applications (2018): 1-12; Fernandes, Kelwin, Jaime S. Cardoso, and Jessica Fernandes. “Temporal segmentation of digital colposcopies.” Iberian conference on pattern recognition and image analysis. Springer, Cham, 2015.)

In some embodiments, in order to train a robust segmentation algorithm for the detection of acetowhite regions from expert-dependent subjective annotations, the following training strategy may be used:
A segmentation deep neural network (DNN) is trained to recognize regions that do not stain using Lugol's iodine;
a post-acetowhitening image and an image with Lugol's iodine are aligned using, e.g., the cervical os as a reference; and
a segmentation DNN is trained to predict which regions do not stain with Lugol's iodine on post-acetowhitening images. Since Lugol's iodine is oversensitive, these regions will include aceto-whitened regions.

The three previous models (i.e., Lugol's segmentation, coarse acetowhite segmentation based on Lugol's annotations, fine acetowhite segmentation) can be used during the inference of a new session by combining the detections from the three models. (See, e.g., Fernandes, Kelwin, and Jaime S. Cardoso. “Ordinal image segmentation using deep neural networks.” 2018 International Joint Conference on Neural Networks (IJCNN). IEEE, 2018).

In some embodiments, system 100 may employ interpretability techniques from deep neural networks (DNN), such as the iNNvestigate (https://github.com/albermax/innvestigate), which does not require training a segmentation network.

hAs will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a hardware processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowcharts and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the description and claims of the application, each of the words “comprise” “include” and “have”, and forms thereof, are not necessarily limited to members in a list with which the words may be associated. In addition, where there are inconsistencies between this application and any document incorporated by reference, it is hereby intended that the present application controls.

1. A method for automated image capture of a body tissue in situ, comprising:
providing an imaging device configured to transmit an image stream of a body tissue; and
using at least one hardware processor for:
receiving the image stream,
identifying a medical accessory appearing in the image stream, and
capturing multiple images of the body tissue, wherein:
at least one of the images is captured before said identifying, and
(ii) at least one of the images is captured at one or more specified times upon said identifying.

2. The method of claim 1, wherein said body tissue is a cervical tissue.

3. The method of claim 1, wherein said medical accessory is a swab used for applying a contrast agent to the body tissue.

4. The method of claim 3, wherein said specified times are determined based, at least in part, on a type of said contrast agent.

5. The method of claim 4, wherein said contrast agent is acetic acid, and said specified times are within the range of 15-600 seconds.

6. The method of claim 5, wherein said range is 90-150 seconds.

7. The method of claim 1, wherein said identifying comprises one or more of: determining a presence of said medical accessory in said image stream for a specified period of time; determining a removal of said medical accessory from said image stream; determining a distance of said medical accessory from the imaging device; determining a specified position of said medical accessory in relation to said body tissue; determining a specified orientation of said medical accessory in relation to said body tissue; identifying a specified object appearing in the image stream simultaneously with said medical accessory; tracking said medical accessory to identify a specified action thereof; and detecting contact between said medical accessory and said body tissue.

8. The method of claim 1, wherein said identifying is executed by a convolutional neural network (CNN) classifier.

9. The method of claim 8, wherein said CNN classifier is trained on a set of labeled images of one of more types of said medical accessory shown against a background of cervical tissue.

10. The method of claim 1, wherein said specified times are determined based on user selection.

11. The method of claim 1, wherein at least some of said multiple images are captured during a specified time period before said specified times, and wherein at least some of said multiple images are captured during a specified time period after said specified times.

12. The method of claim 1, wherein said capturing comprises capturing one or more of a continuous video sequence, a time-lapse video sequence, and a sequence of images.

13. The method of claim 12, wherein said capturing further comprises selecting an image from the sequence of images based at least in part on an image quality parameter.

14-20. (canceled)

21. A system comprising:
an imaging device configured to transmit an image stream of a body tissue;
at least one hardware processor; and
a non-transitory computer-readable storage medium having stored thereon program instructions, the program instructions executable by the at least one hardware processor to automatically:
receive the image stream,
identify a medical accessory appearing in the image stream, and
capture multiple images of the body tissue, wherein;
(i) at least one of the images is captured before said identifying, and
(ii) at least one of the images is captured at one or more specified times upon said identifying.

22-75. (canceled)

76. The system of claim 21, wherein said body tissue is a cervical tissue.

77. The system of claim 21, wherein said medical accessory is a swab used for applying a contrast agent to the body tissue.

78. The system of claim 77, wherein said specified times are determined based, at least in part, on a type of said contrast agent.

79. The system of claim 78, wherein said contrast agent is acetic acid, and said specified times are within the range of 15-600 seconds.

80. The system of claim 21, wherein said identifying comprises one or more of:
determining a presence of said medical accessory in said image stream for a specified period of time; determining a removal of said medical accessory from said image stream; determining a distance of said medical accessory from the imaging device; determining a specified position of said medical accessory in relation to said body tissue; determining a specified orientation of said medical accessory in relation to said body tissue; identifying a specified object appearing in the image stream simultaneously with said medical accessory; tracking said medical accessory to identify a specified action thereof; and detecting contact between said medical accessory and said body tissue.

81. The system of claim 21, wherein said identifying is executed by a convolutional neural network (CNN) classifier that is trained on a set of labeled images of one of more types of said medical accessory shown against a background of cervical tissue.
